(19) United States
(12) Patent Application Publication
(10) Pub. No.: US 2007/0111225 A1
(43) Pub. Date: May 17, 2007

(54) SYSTEM AND METHOD FOR MONITORING AN ANALYTE

(75) Inventors: James L. Lambert, Sunland, CA (US); Anita M. Fisher, La Crescenta, CA (US); Mark S. Borchert, La Canada, CA (US)

Correspondence Address: DAVIS WRIGHT TREMAINE LLP, 865 FIGUEROA STREET, SUITE 2400, LOS ANGELES, CA, 90017-2566, US

(73) Assignee: CALIFORNIA INSTITUTE OF TECHNOLOGY, Pasadena, CA (US)

(21) Appl. No.: 11/463,834

(22) Filed: Aug. 10, 2006

Related U.S. Application Data
(60) Provisional application No. 60/706,960, filed on Aug. 10, 2005.
(60) Provisional application No. 60/750,534, filed on Dec. 15, 2005.

(51) Int. Cl. C12Q 1/68; G01N 33/53; G06F 19/00

(52) U.S. Cl. 435/6; 435/7.1; 702/19; 702/21; 977/924

(57) ABSTRACT

The present invention provides for a monitoring system and method to monitor an analyte, including determining the presence of the analyte and analyzing the analyte; for example, cellular chorography and biological threats. The system and method use a fluidics apparatus (e.g., a flow cytometer), a computer, a probe synthesizer and/or an initial set of fluorophore conjugated probes to monitor the analyte. The fluidics apparatus is adapted to determine the presence of the analyte based on the intrinsic fluorescence of the analyte, and/or to determine the binding of the probes to the analyte in the sample. The binding information is analyzed by the computer by comparing the binding results with a database of information regarding the analyte. The information gained allows the probe synthesizer to synthesize new probes for a subsequent iteration of the process, wherein additional information regarding the analyte is gained with every iteration.

This application claims priority to U.S. provisional application Ser. No. 60/706,960, filed Aug. 10, 2005, and U.S. provisional application Ser. No. 60/750,534, filed Dec. 15, 2005.

### GOVERNMENT RIGHTS

The invention described herein was made in the performance of work under a NASA contract, and is subject to the provisions of Public Law 96-517 (35 U.S.C. §202) in which the Contractor has elected to retain title.

### FIELD OF INVENTION

This invention relates a system and method for monitoring an analyte, including determining the presence of the analyte and/or analyzing the analyte. The system may be used to monitor and analyze a variety of analytes; for example, cellular chorography, microorganism phenology and biological threats.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Scientists currently use series of biochips to analyze the DNA, RNA, or proteins produced, to follow complex cellular chorography. In developmental biology, probes for mRNA templates in cells are used to examine the type of mRNA or protein expression in which the cell is currently engaged. Since the mRNA's expression causes other mRNA's to be expressed, a series of biochip assays are often used in conjunction with bioinformatics software to try to follow the complex multi-step processes within cells.

However, the use of a series of biochip assays can be a slow process and can necessitate many separate steps to achieve the analysis. Thus, there is a need for a system and a method to more efficiently analyze the complex cellular chorography, and to streamline and quicken this process.

Additionally, microbial contaminants have been identified in the international space station (ISS) and Mir water supplies. These contaminants include, but are not limited to, Aeromonas species, Agrobacterium rhizogenes, Bacillus licheniformis, Bacillus macerans, Bacillus polymyxa, Bacillus species, Burkholderia cepacia, Burkholderia pickettii, CDC Group EF4, CDC Group II-H, CDC Group IVC-2, Clavibacter michiganense, Corynebacterium aquaticum, Corynebacterium jeike, Corynebacterium species, Enterobacter georgiae, Flavobacterium species, Flavobacterium meningosepticum, Hydrogenophaga pseudoflava, Kingella denitrificans, Kingella kingae, Kingella species, Kluvera ascorbata, Flavobacterium indologenes, Methylobacterium extorquens, Methylobacterium species, Micrococcus kristinae, Micrococcus species, Pseudomonas aeruginosa, Pseudomonas fluorescens, Pseudomonas vesicularis, Psychrobacter glathei, Rhizobium loti, Sphingobacterium thalpophilium, Sphingomonas paucimobilis, Staphylococcus aureus, Staphylococcus capitis, Staphylococcus epidermidis, Suttonella indologenes, Xanthomonas campestris, Xanthomonas maltophilia, and Xanthomonas species.

The U.S. Centers for Disease Control and Prevention (CDC) have also identified known bioterrorism agents. The agents include but are not limited to Anthrax (Bacillus anthracis), Botulism (Clostridium botulinum toxin), Brucella species (brucellosis), Burkholderia mallei (glanders), Burkholderia pseudomallei (melioidosis), Chlamydia psittaci (psittacosis), Cholera (Vibrio cholerae), Clostridium perfringens (Epsilon toxin), Coxiella bumetii (Q fever), E. coli O157:H7 (Escherichia coli), Emerging infectious diseases (e.g., Nipah virus/hantavirus), Food safety threats (e.g., Salmonella species, Shigella, E. coli), Francisella tularensis (tularemia), Plague (Yersinia pestis), Ricin toxin (from Castor Beans), Rickettsia prowazekii (typhus fever), Salmonella Typhi (typhoid fever), Salmonellosis (Salmonella species), Shigella (shigellosis), Smallpox (variola major), Staphylococcal enterotoxin B, Viral encephalitis (alphaviruses [e.g., Venezuelan equine encephalitis, eastern equine encephalitis, western equine encephalitis]), Viral hemorrhagic fevers (filoviruses [e.g., Ebola, Marburg] and arenaviruses [e.g., Lassa, Machupo]), and Water safety threats (e.g., Vibrio cholerae, Cryptosporidium parvum).

With the existence of known and emerging biological threats in water, there is also a need to continuously monitor potable water supplies for known and unknown biological agents. A method and system for screening large volumes of water without the need for consumables is desirable since this would vastly reduce the cost of operation. If a biological threat is detected, there is a need to identify it as: (i) harmless; (ii) a strain of a known pathogen; or (iii) an unknown bacterial strain, but related to one or more known species.

### SUMMARY OF THE INVENTION

The following embodiments and aspects thereof are described and illustrated in conjunction with systems and methods are meant to be exemplary and illustrative, not limiting in scope.

Embodiments of the present invention provide for methods for monitoring an analyte in a sample, comprising providing the sample; and detecting a presence of the analyte in the sample, wherein detecting the presence of the analyte comprises, placing the sample through a fluidics apparatus, using a laser to excite an intrinsic fluorophore in the analyte, detecting a fluorescence in the sample, and comparing the detected fluorescence to a database of information, including information on the fluorescence of the intrinsic fluorophore in the analyte, wherein if the detected fluorescence corresponds to the fluorescence of the intrinsic fluorophore, the analyte is determined to be present in the sample. In one embodiment, the fluidics apparatus is a flow cytometer.

In one embodiment, the analyte may be a microorganism or a cell. In another embodiment, the sample may be water or a physiological fluid.

In various embodiments, the laser may operate at a wavelength of from about 220 nm to about 240 nm or from about 270 nm to about 290 nm and detecting the fluorescence may comprise detecting a fluorescence signal at from about 320 nm to about 370 nm. In one embodiment, the laser may operate at a wavelength of about 227 nm or about 280 nm, and detecting the fluorescence may comprise detecting a fluorescence signal at about 340 nm.

In one embodiment, the method further comprises collecting the analyte by electrostatically deflecting a portion of the sample containing the analtye into a container. In various embodiments, different analytes may be defected into different containers.

In another embodiment, the method further comprises analyzing the analyte. Analyzing the analyte may comprise using a fluorophore-conjugated immunoassay method or a fluorophore-based adaptive analysis method. In various embodiments, the fluorophore may be a quantum dot or a quantum bead.

In one embodiment, the immunoassay method may comprise using a fluorophore-conjugated probe capable of binding to a particular analyte; and determining the binding of the fluorophore-conjugated probe to the analyte, wherein the binding of the fluorophore-conjugated probe to the analyte indicates the presence of the particular analyte.

In one embodiment, determining the binding of the fluorophore-conjugated probe may comprises using a laser at a wavelength capable of exciting the fluorophore; and detecting the emission of the fluorophore and a scattering of light by the analyte, wherein the detection of both the emission of the flurorphore and the scattering of the light indicate the binding of the probe to the analyte.

In one embodiment, the probe may be an antibody or an aptamer.

In one embodiment, the fluorophore-based adaptive analysis method may comprise providing an initial fluorophore conjugated probe set; adding the probe set to the sample; determining the binding of the probe to the analyte to produce binding results; comparing the binding results to a database of information regarding the analyte; generating a new fluorophore conjugated probe set based on the comparison; and repeating the process with the new fluorophore conjugated probe set until the desired analysis of the analyte is performed.

In various embodiments, the probe may be deoxyribonucleic acid (DNA), ribonucleic acid (RNA), peptide nucleic acid (PNA), aptamer, peptide, antibody or combinations thereof.

In one embodiment, determining the binding of the probe to the analyte may comprise using a laser at a wavelength capable of exciting the fluorophore; and detecting the emission of the fluorophore and a scattering of light by the analyte, wherein the detection of both the emission of the flurorphore and the scattering of the light indicate the binding of the probe to the analyte.

Further embodiments of the present invention provides for methods for adaptive analysis of an analyte in a sample comprising providing an initial fluorophore conjugated probe set; adding the probe set to the sample; determining the binding of the probe to the analyte to produce binding results; comparing the binding results to a database of information regarding the analyte; generating a new fluorophore conjugated probe set based on the comparison; and repeating the process with the new fluorophore conjugated probe set until the desired monitoring or analysis of the analyte is performed.

In various embodiments, the probe may be deoxyribonucleic acid (DNA), ribonucleic acid (RNA), peptide nucleic acid (PNA), aptamer, peptide, antibody or combinations thereof. In a particular embodiment, the probe is a peptide nucleic acid (PNA). In another embodiment, the probe is an aptamer.

In various embodiments, the analyte may be a microorganism or a cell. In other embodiments, the sample may be water or a physiological fluid. In various embodiments the fluorophore may be a quantum dot or a quantum bead.

In one embodiment, determining the binding of the probe to the analyte may comprise using a laser at a wavelength capable of exciting the fluorophore; and detecting the emission of the fluorophore and a scattering of the light by the analyte, wherein the detection of both the emission of the fluororphore and the scattering of the light indicate the binding of the probe to the analyte.

Additional embodiments of the present invention provide for systems for monitoring an analyte in a sample, comprising a fluidics apparatus adapted to create a fluid stream of the sample and/or to create a series of small drops of the sample; a laser adapted to operate at a wavelength that is capable of inducing an intrinsic fluorescence of the analyte; a counting component adapted to determine the concentration of the analyte; an reporting component adapted to report the concentration of analyte that is above or below a predetermined concentration; and a sorting component adapted to apply a charge to a portion of the sample containing the analyte and to deflect the charged portion of the sample containing the analyte into a container. Based on te fluorescence detected, different analytes may be deflected into different containers. In one embodiment, the fluidics apparatus is a flow cytometer. In various embodiments, the analyte may be a microorganism or a cell.

Further embodiments of the present invention provide for adaptive analysis systems for analysis of an analyte in a sample, comprising a fluidics apparatus adapted to determine the binding of a fluorophore conjugated probe to the analyte; a probe synthesizer to synthesize a new set of fluorophore conjugated probes; and a computer adapted to analyze the binding of the probes, compare the binding of the probes to a database of information regarding the analyte, and provide information to the probe synthesizer regarding the type of probes to synthesize. In a further embodiment, the system may further comprise a set of fluorophore conjugated probes adapted to selectively bind to analytes that may be present in the sample. In various embodiments, the analyte may be a microorganism or a cell. In various embodiments, probe may be deoxyribonucleic acid (DNA), ribonucleic acid (RNA), peptide nucleic acid (PNA), aptamer, peptide, antibody or combinations thereof. In various embodiements the fluorophore may be a quantum dot or a quantum bead.

Further embodiments of the present invention provide for methods of phylogenetic classification of a microorganism in a sample, comprising providing an initial fluorophore conjugated probe set that is complementary to a 16S ribosomal ribonucleic acid (rRNA) sequence; adding the probe set to the sample; determining the binding of the probe to rRNA in the microorganism to generate binding results; comparing the binding results to a database of information regarding microorganisms; generating a new fluorophore conjugated probe set based on the comparison; and repeating the process with the new fluorophore conjugated probe set until the desired level of phylogenetic classification of the microorganism is performed. In one embodiment, the probe is a peptide nucleic acid (PNA). In various embodiments, the fluorophore may be a quantum dot or a quantum bead.

Still further embodiments of the present invention provide for methods for adaptive production of a therapeutic compound, comprising providing an initial set of fluorophore conjugated therapeutic compounds capable of binding to an analyte for which the therapeutic compound is to be produced; adding the set of fluorophore conjugated therapeutic compounds to a sample comprising the analyte; determining the binding of the therapeutic compounds to the analyte to generate binding results; comparing the binding results to determine a similarity between bound therapeutic compounds and/or comparing the binding results to a database of information regarding the analyte; generating a new set of fluorophore conjugated therapeutic compounds based on the comparison; and repeating the process with the new set of fluorophore conjugated therapeutic compounds until the desired therapeutic compound is synthesized. The therapeutic compound may comprise deoxyribonucleic acid (DNA), ribonucleic acid (RNA), peptide nucleic acid (PNA), aptamer, peptide, antibody or combinations thereof. The fluorophore may be a quantum dot or a quantum bead.

In various embodiments of the present invention, the physiological fluid may be interstitial fluid, saliva, sweat, urine, whole blood, serum, plasma, cerebral spinal fluid (CSF), tears, pulmonary secretion, breast aspirate, prostate fluid, seminal fluid, amniotic fluid, intraocular fluid, mucous or combinations thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are considered illustrative rather than restrictive.

FIG. 1 depicts UV Excitation Emission Matrices (EEM) of Archaea, Bacteria, and their intrinsic fluorophores in accordance with an embodiment of the present invention. Vertical and horizontal axes of EEM's indicate excitation and emission wavelengths (nm), respectively.

FIG. 2 depicts UV EEM of minerals, particulates, and organic soil components (polyaromatic hydrocarbon compounds) in accordance with an embodiment of the present invention. Vertical and horizontal axes of EEM's indicate excitation and emission wavelengths (nm), respectively.

FIG. 3 depicts a flow diagram of a bioagent detection and identification system in accordance with an embodiment of the present invention.

FIG. 4 depicts intrinsic fluorescence as shown on the contour map of an emission-excitation matrix of 2 μg/mL of tryptophan in distilled water in accordance with an embodiment of the present invention. The vertical axis shows the excitation wavelengths and the horizontal axis shows the emission wavelengths. The emission spectra from 295 nm-500 nm for every excitation wavelength from 200-290 nm in steps of 2 nm. The fluorescence emission reaches a maximum at 360 nm when the excitation is either ˜220 nm or ˜280 nm.

FIG. 5 depicts microbial screening of 300 year-old Greenland Melt-Ice in accordance with an embodiment of the present invention. (A) UV EEM data of water showing water Raman feature (pure water); (B) UV EEM data of low-level contamination (tap water); (D) UV EEM data of GISP2 ice core; (C) the excitation peaks at 230 nm and 280 nm with peak emission at 330 nm characteristic of tryptophan signature in bacteria (e.g., Bacillus subtilis). Microbial life was confirmed using flow cytometry of the GISP2 melt-ice (E, F).

FIG. 6 depicts multiplexed assays with quantum dots in accordance with an embodiment of the present invention. (A) Excitation of CdSe/ZnS quantum dots; (B) Excitation Spectrum of CdSe/ZnS quantum dots; (C) Emission Spectrum of CdSe/ZnS quantum dots.

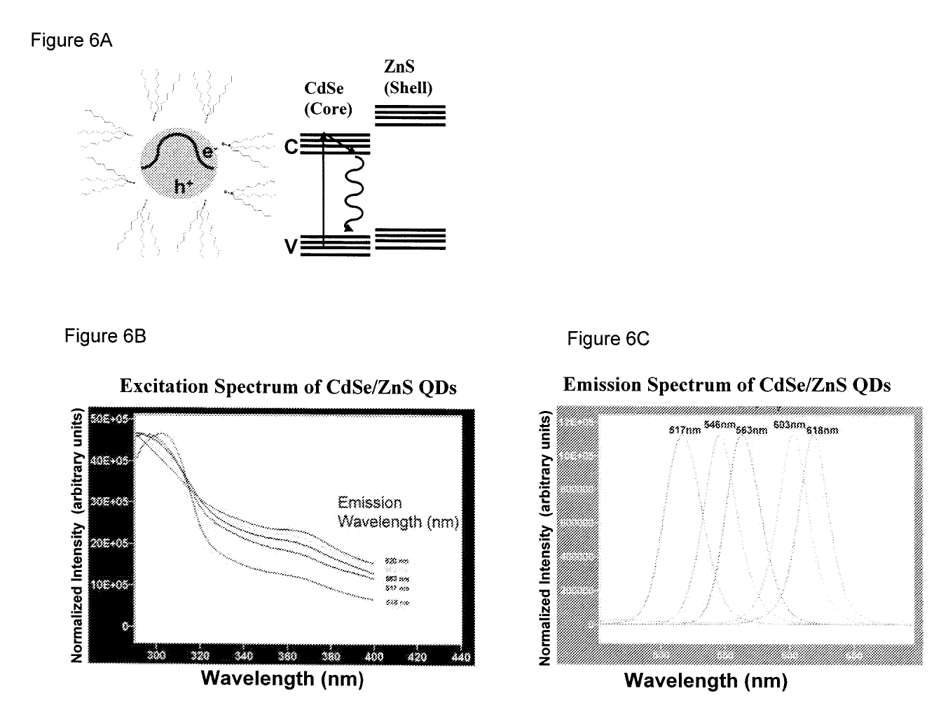

FIG. 7 depicts a flow diagram of adaptive biosensing to detect bacterial threats in accordance with an embodiment of the present invention.

FIG. 8 depicts rRNA phylogenetic classification in accordance with an embodiment of the present invention.

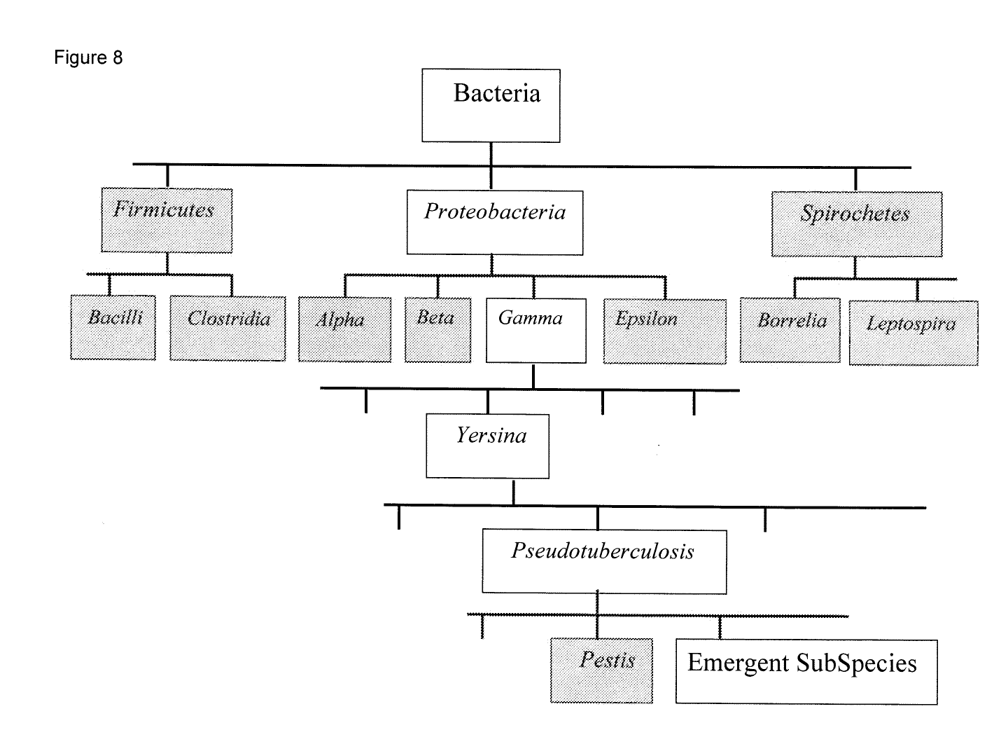

FIG. 9 depicts an EEM of tryptophan in accordance with an embodiment of the present invention.

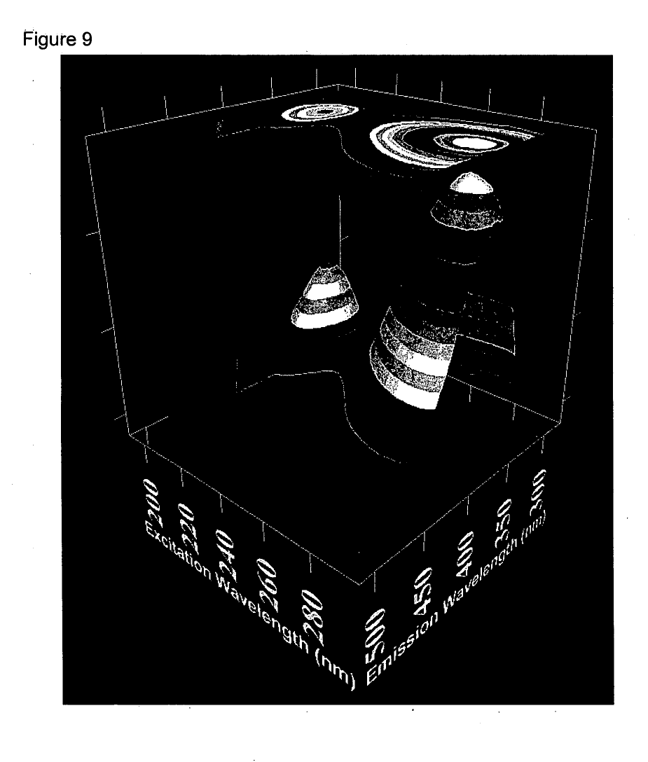

FIG. 10 depicts classification of particles using one or more fluorophores in accordance with an embodiment of the present invention. (A) a diameter of the microbial contaminant is greater than the wavelength of light and thus scatters light, but there will be no fluorescence at visible wavelengths because no QD-tagged antibodies are bound; (B) a QD-tagged antibody will fluoresce in the visible wavelength, but since it is not bound to a microbial contaminant, it is too small to scatter light from the excitation beam; (C) The microbial contaminants with bound QD-tagged antibodies will scatter light and fluoresce allowing the identity of the contaminant to be determined.

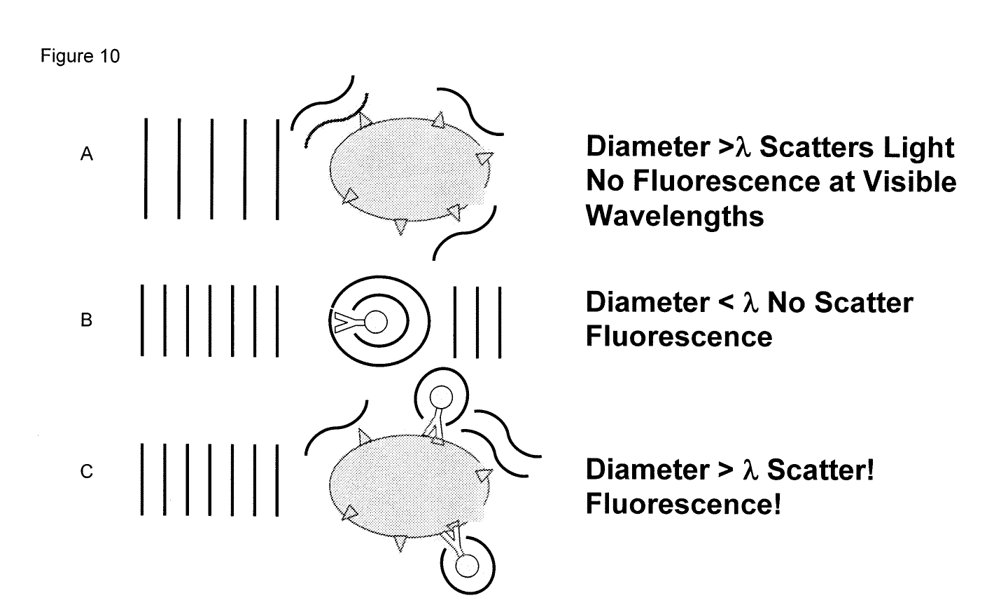

FIG. 11 depicts elastic scattering and fluorescence emission spectrum in accordance with an embodiment of the present invention.

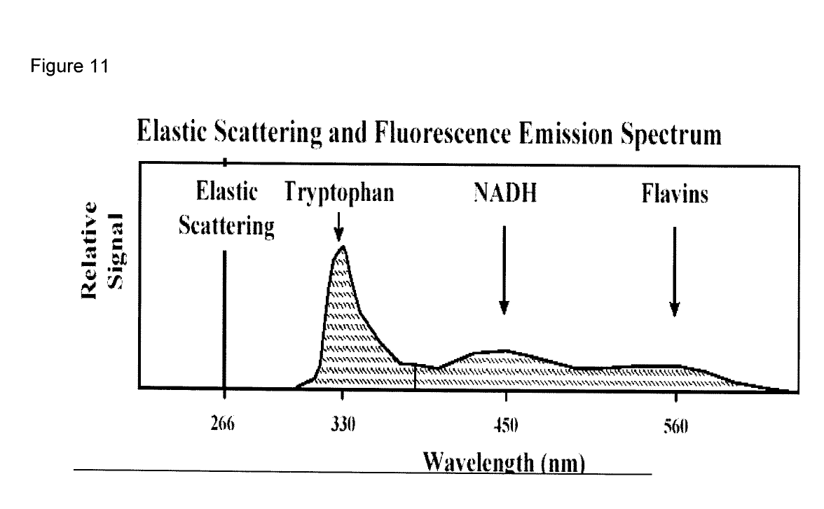

FIG. 12 depicts an emission spectrum taken with an excitation wavelength of 275 nm of a mixture of four sizes of CdSe/ZnS quantum dots in accordance with an embodiment of the present invention. Four distinct peaks at 525 nm, 570 nm, 605 nm, and 650 nm are present. Quantum dots have the property that a single excitation wavelength can cause all of the fluorophores to fluoresce simultaneously. Additionally in the UV the CdSe quantum dots have extinction coefficients on the order of 2.8 E6/cm/M and quantum yield of at least 50%. Therefore, CdSe quantum dots have a brightness value of nearly 1,400,000. For example fluorescein, one of brightest organic dyes, has brightness value of 64,000.

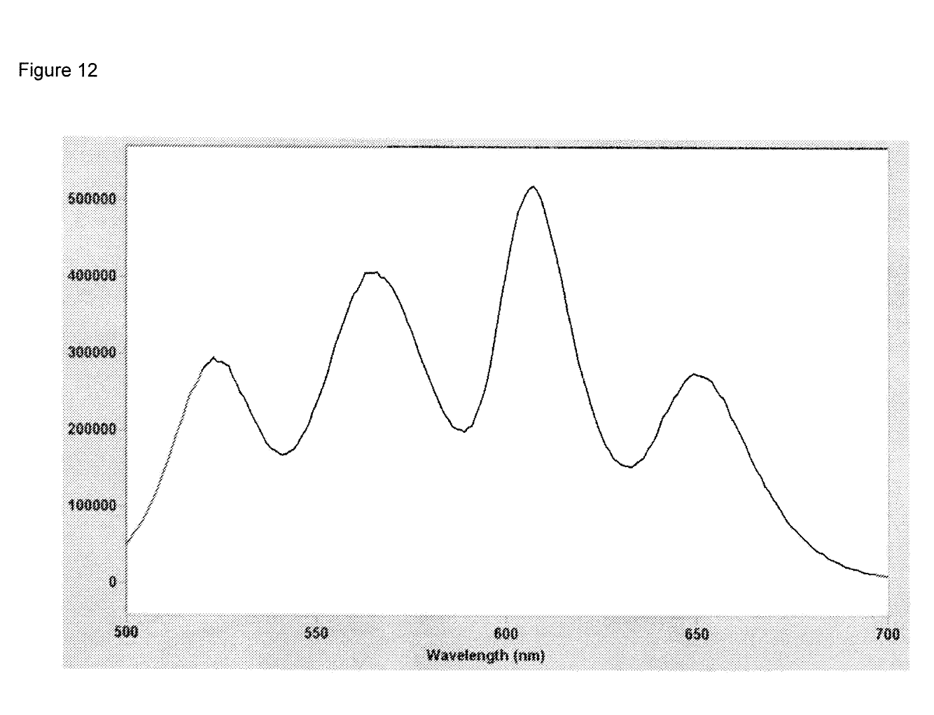

FIGS. 13A and 13B depict the EEM of Bacillus Cereus (Raven Biological) and Burkholderia spp. (Atacama Desert), respectively, in accordance with embodiments of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Analyte” as used herein refers a substance, chemical, chemical constituent, biological process, or biological component that is undergoing analysis; for example, microorganisms, mRNA expression, surface cell markers, proteins, nucleic acids of any type (e.g., DNA, RNA), drugs, chemical compounds, etc.

“Aptamer” as used herein refers to an oligonucleic acid or peptide molecule that is capable of binding to a specific target molecule. The aptamer may be a DNA, RNA or peptide aptamer.

“Emerging” or “emergent,” in reference to bacteria, microorganisms, subspecies, and the like, as used herein refer to strains of bacteria, microorganisms, etc. that have not been previously classified, or mutations of classified strains of bacteria, microorganisms, etc.

“Quantum dot,” also commonly referred to as nanocrystals or semiconductor nanocrystals, as used herein refers to a semiconductor crystal whose size is on the order of nanometers. The energy levels of a quantum dot can be controlled by changing the size and shape of the quantum dot, and the depth of the potential. The energy levels of small quantum dots can be probed by optical spectroscopy techniques. During fabrication, the diameter of quantum dots can be selected to achieve emission fluorescence in a variety of colors.

“Therapeutic agent” as used herein refers to agents capable of treating a disease condition; for example, chemotherapeutic drugs. Additional examples of therapeutic agents include: therapeutic viral particles, antimicrobials (e.g., antibiotics, antifungals, antivirals), and antibodies. Other suitable therapeutic agents will be readily recognized by those of skill in the art.

“Treatment” and “treating,” as used herein refer to both therapeutic treatment and prophylactic or preventative measures, wherein the object is to prevent or slow down (lessen) the targeted pathologic condition or disorder even if the treatment is ultimately unsuccessful. Those in need of treatment include those already with the disorder as well as those prone to have the disorder or those in whom the disorder is to be prevented. For example, in cancer treatment, a therapeutic agent may directly decrease the pathology of tumor cells, or render the tumor cells more susceptible to treatment by other therapeutic agents or by the subject's own immune system.

A system and a method for monitoring an analyte, including the detection of an analyte, analysis of the analyte and/or adaptive analysis of an analyte are described. The system and method use liquid based assaying. The analyte may be any number of things. Examples of analytes include but are not limited to microorganisms; nucleic acids, proteins or peptides within microorganisms; cells; and nucleic acids, proteins or peptides within cells.

The inventive system comprises a fluidics apparatus adapted to determine the presence of an analyte in the sample. The fluidics apparatus may be a flow cytometer. The fluidics apparatus may comprise a laser adapted to operate at a wavelength that is capable of inducing an intrinsic fluorescence of the analyte and a sorting component adapted to apply a charge to a portion of the sample containing the analyte and then to deflect the charged portion of the sample containing the analyte into a container, in which the analyte may be analyzed. In various embodiments, different analytes may be defected into different containers based on the intrinsic properties detected, such as the intrinsic fluorescence detected. An analyte may have an intrinsic fluorescence and thus an emission at a particular wavelength, along with the scattering of light, will indicate the presence of the analyte. Other intrinsic properties of the analyte may be used to detect the presence of the analyte; for example, scattering, the morphology of the analyte (e.g., shape, size), absorption, dielectric constant. One skilled in the art will recognize other intrinsic properties of the analyte for which its detection may be based. The particular wavelength depends on the analyte that is being monitored or analyzed by the system.

In one embodiment, the adaptive analysis system may include an initial set of fluorophore conjugated probes, a fluidics apparatus (e.g., flow cytometer) a computer and a probe synthesizer. The set of fluorophore conjugated probes may be adapted to selectively bind to analytes that may be present in a sample that is placed through the system. The fluidics apparatus (e.g., flow cytometer) may be adapted to determine the binding of the probes to the analyte in the sample. In various embodiments, the fluidics apparatus may be a simple flow through apparatus. In other embodiments, the fluidics apparatus may comprise multiple chambers. In other embodiments, the fluidics apparatus may have one or more jets to stream the liquid and/or may be cuvet based. The binding of the probes to the analyte may be determined by using a laser to excite the fluorophore that is conjugated to the probe. An emission at a particular wavelength along with a scattering of light indicates the binding of the probe to the analyte. The particular wavelength depends on the fluorophore used. The computer may be adapted to analyze the binding of the probes to the analyte in the sample by utilizing an algorithm to compare the binding results to a database. The analysis of the binding dictates the type of probes that are synthesized by the probe synthesizer for a subsequent iteration to gain additional information regarding the analyte. The probe synthesizer is adapted to receive information from the computer and synthesize a new set of fluorophore conjugated probes for a subsequent iteration. The system may be configured to repeat this process for any desired number of iterations.

The present invention also provides for methods for monitoring including detection of an analyte, analysis of an analyte and/or adaptive analysis of an analyte in a sample. In one embodiment, the inventive method includes the step of providing an initial set of fluorophore conjugated probes that are capable of selectively binding to an analyte that may be present in a sample. The set of probes may be added to the sample to allow for any binding to occur. After an appropriate amount of time, which depends on the analyte being monitored or analyzed as well as other factors readily recognizable by those skilled in the art, the sample may be run through a fluidics apparatus (e.g., flow cytometer) to determine the binding of the probes to the analytes. The flow cytometer may utilize a laser to excite the fluorophores that are conjugated to the probes. A detection of fluorescence at a particular wavelength along with the scattering of light by the analyte may indicate probe binding. The particular wavelength is dependent upon the fluorophore that is conjugated to the probe. The binding information may be processed by a computer that utilizes an algorithm that compares the binding information to a database to dictate the type of probes that are synthesized by a probe synthesizer for the next iteration. Alternatively, the binding information may be used to select the next set of pre-made probes that are used for the next iteration. Once the next set of probes is synthesized or selected, the probes may be added to a fresh sample or the same sample and the process may be repeated until the desired monitoring or analysis of the analyte is performed.

A flow cytometer may be used to sort a sample containing an analyte such as a microorganism. A flow cytometer works by placing a fluidic sample into a nozzle and surrounding this nozzle by a funnel shaped vessel where clean fluid (called a sheath fluid) is injected. Gaseous nitrogen may be used to force both liquids through both of these chambers at different pressures. The sheath fluid may be used to “hydrodynamically focus” the sample fluid into a cylindrically shaped stream. The higher the differential pressure of the fluids, the narrower the sample stream becomes. One or more lasers may be used to identify and classify particles flowing through the sample stream based upon their scattering and fluorescence properties. Particles larger than the wavelength of laser light illuminating the stream will scatter light. The light scattered in the same direction as the laser beam (forward scatter) is often used to indicate that a particle is in the stream and can be used as an indication of the physical size of the particle. Light scatter at some angle from the laser beam (side scatter) can be used as an indication of the texture of particles. Fluorescent dyes are often used to selectively identify particles based on their characteristics. In addition to the dyes, various embodiments of the present invention use quantum dot conjugated probes or quantum bead conjugated probes. Photomultiplier tubes with filters tuned to the emission peaks of these dyes may be used to measure the fluorescent signals in real time. Electronics may be used to digitize the signals from the forward scatter, side scatter, and fluorescence detectors. The flow cytometer has the ability to separate those particles having specific scatter and fluorescence signatures in real time. A piezoelectric crystal may be used to vibrate the sample nozzle so that downstream from the laser fluorescence and scatter measurement, the stream is broken into a series of small drops each containing a single particle, which may be the analyte. The sample nozzle may be used to place a charge on these droplets and electrostatic plates may be used to direct those particles with the desired fluorescence and scattering characteristics into one or more containers for analysis. The system may be adapted to have mirrors coated to pass UV light and filters to detect wavelengths of about 1 nm to about 1000 nm. In one embodiment the filters may be adapted to detect wavelengths of about 200 nm to about 500 nm. In another embodiment, the filters may be adapted to detect wavelengths of about 300 nm to about 400 nm.

While flow cytometry is a well known field, the present invention uses flow cytometry in a novel and unobvious way to detect an analyte and/or to specifically identify or analyze the analyte. For example, in one embodiment of the present invention, a flow cytometer may be used to first count bacteria based on their intrinsic fluorescence. This may be followed with an assay to analyze the analyte; for example in instances where the analyte is a microorganism, the strain(s) of bacteria captured may be determined. The assay may be a quantum dot-based assay; for example, an immunoassay or an adaptive monitoring assay. Embodiments of the present invention use intrinsic fluorescence of bacteria to continuously monitor water that is presumed fairly free of bacterial contamination for microbial contamination. Other embodiments of the present invention may be used to monitor spacecraft drinking and process water, and municipal water supplies, or to search for life in the water-ice or aqueous terrestrial or extraterrestrial environments (e.g., Antarctic ice, Mars polar caps or European ice or oceans, etc.).

The probes that are synthesized or used by the system and method may comprise deoxyribonucleic acids (DNA) ribonucleic acids (RNA), peptide nucleic acids (PNA), aptamers, peptides or antibodies.

The fluorophore conjugated to the probes may be a quantum dot, quantum bead, or nanoparticles for surface enhance Ramen spectroscopy (e.g., metal nanoparticles such as gold and silver) (see., e.g., M. Moskovits, Surface-Enhance Raman spectroscopy: a brief retrospective. J. Raman. Spec., 2005, 36, 485-496). Other fluorophores include fluorescein isothiocyanate (FITC) and Texas Red (a sulfonyl chloride derivate of sulforhodamine). Still further fluorophores will be readily known to those of skill in the art and can be used in alternate embodiments of the present invention. In various embodiments, a single wavelength may be used to induce different fluorescence signals and/or signatures given by different fluorophores. In other embodiments, a single wavelength may be used to induce different Ramen spectra given by different nanoparticles used for surface enhanced Ramen Spectroscopy. In order to examine several binding constants in parallel, quantum dots or quantum beads, each with a unique emission spectrum, may be conjugated to the probes. For example, cadmium selenide (CdSe) or CdSe in the core and zinc sulfide (ZnS) in the shell (CdSe-ZnS), quantum dots are commercially available and are easily conjugated to the probes. They may also be specially coated to be water-soluble. A variety of different sized CdSe quantum dots are commercially available that produce specific narrowband (e.g., 30 nm) fluorescence emission across the visible spectral range given excitation from a single wavelength of light (e.g., 352 nm).

### Adaptive Monitoring for Known and Emerging Biological Threats

In one embodiment, intrinsic fluorescence is used to reagentlessly detect a microorganism in water. This may be performed by use of scattering to determine that a particle of some type is present in the sample stream using a scatter signal from a laser. Additionally, a very short wavelength of UV light (e.g., about 230 nm and/or about 280 nm) may be used to determine if the particle has an intrinsic fluorescence signal indicating that the particle contains a significant quantity of the amino acid tryptophan. In various embodiments, a wavelength of from about 220 nm to about 240 nm and/or from about 270 nm to about 290 nm may be used. In one embodiment, the laser may operate at a wavelength of about 227 nm and/or about 280 nm. Tryptophan is a very bright intrinsic fluorophore that is present in most proteins. It is on this basis—the particle scatters light (e.g., the particle is 1 micron in size) and fluoresces at an emission wavelength indicative of tryptophan (about 320 nm)—that it can be concluded that the particle is a microbial contaminant (it may be living or dead). In various embodiments, emission wavelengths of from about 320 nm to about 370 nm may also be indicative of tryptophan. In one embodiment, an emission wavelength of about 340 nm may be indicative of tryptophan. As particles flow through the instrument a count may be performed. Unlike the normal cytometers, intrinsic fluorescence of the particles is used. The particles are not dyed prior to analysis. Because no reagents are used during the detection process, the instrument can work for prolonged periods of time using no consumables and with no operator interaction.

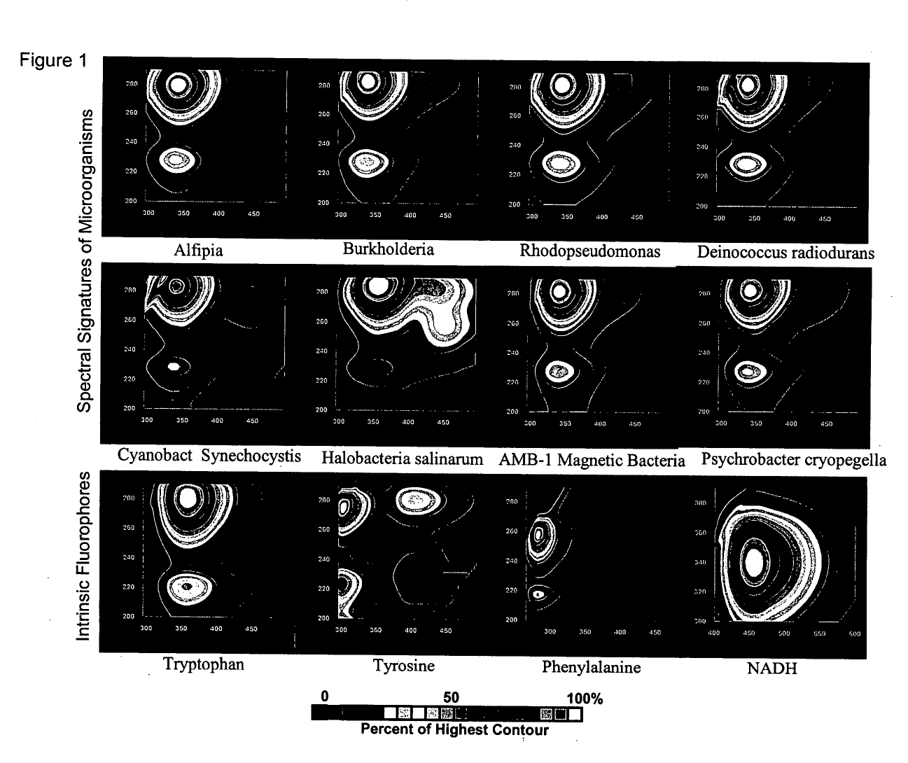

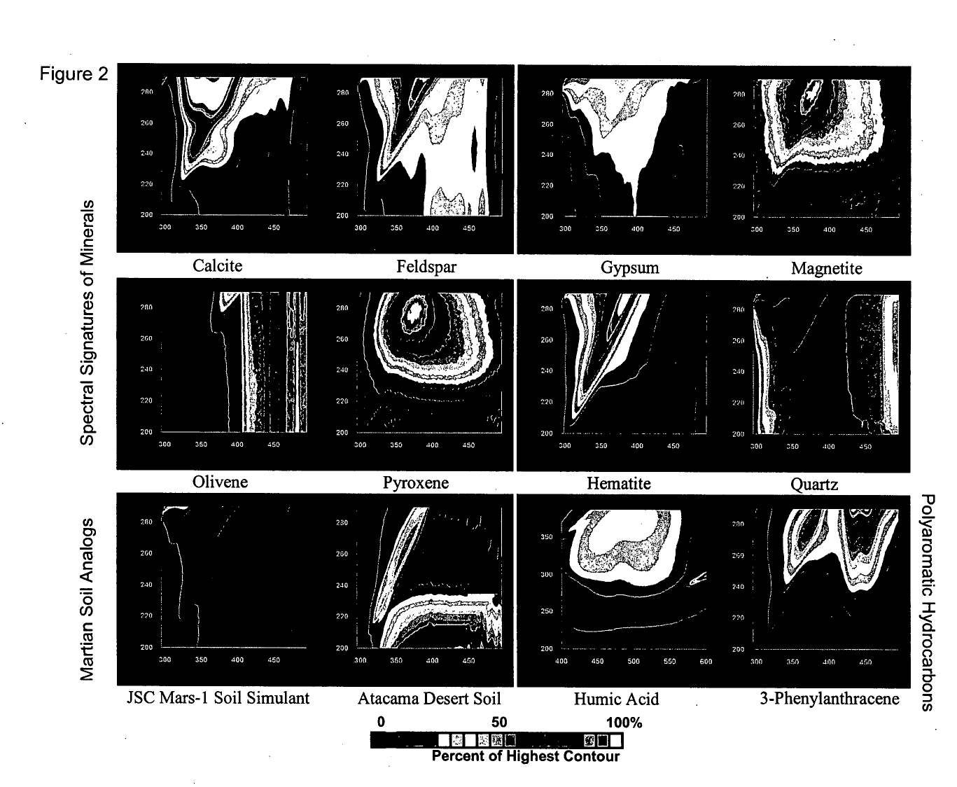

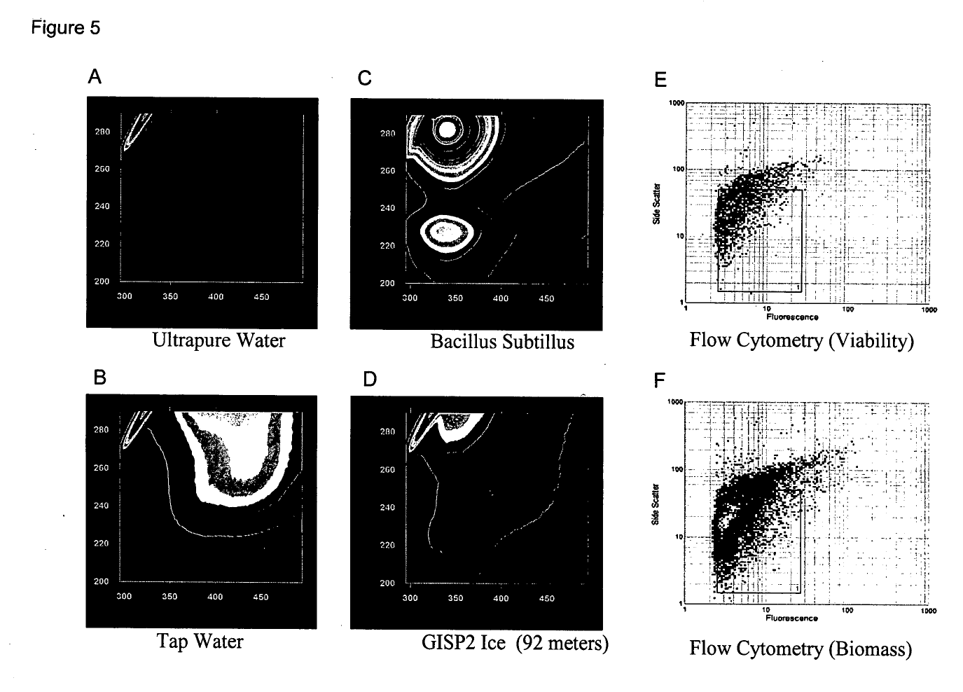

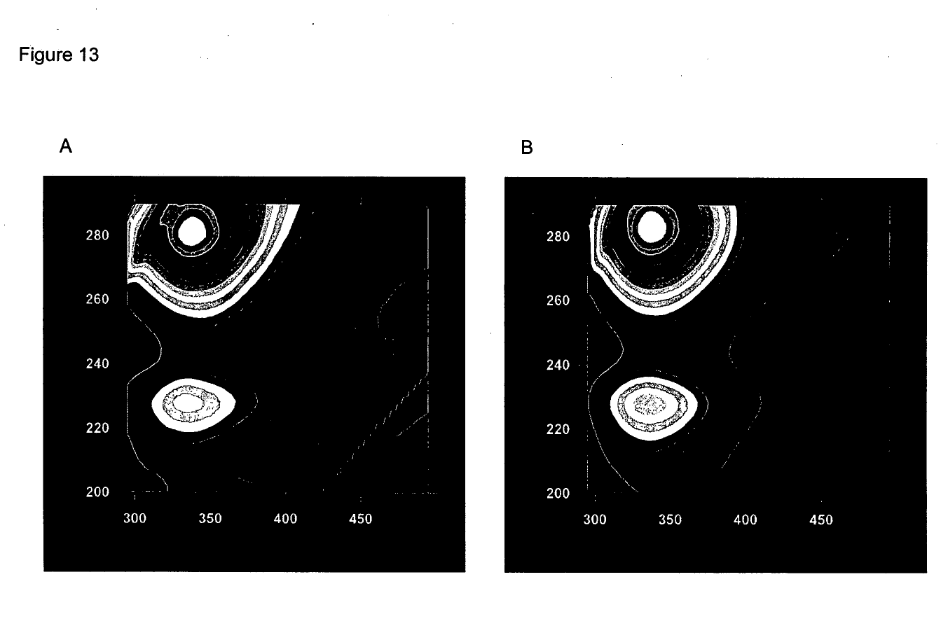

The inventors' measurements as well as others have shown that laser induced fluorescence is exhibited by all bacteria and archaea. The intrinsic fluorophores tryptophan, tyrosine, phenylalanine, NADH (reduced nicotinamide adenine dinucleotide), and FAD (flavin adenine dinucleotide) give rise to a relatively consistent signature that may be used to delineate biogenic particulates as well as other contaminants. When an excitation-emission matrix of a 0.1 OD suspension of Bacillus Subtilis is collected, the characteristic tryptophan signature is readily apparent. However, the peak emission wavelengths are shifted to 340 nm and the 220 nm excitation maxima has shifted from 220 nm to 227 nm. When an excitation-emission matrix from a Bacillus Cereus is collected, a matrix nearly identical to the Bacillus Subtilus is observed. When a completely different kingdom of bacteria is run (Burkholderia spp.), a nearly identical signature is obtained. After running different types of bacteria, the same signature indicative of (shifted) tryptophan is repeated. (See e.g., FIGS. 1, 5C and 13.) Therefore, by using peak excitation wavelengths near 227 nm and 280 nm it appears likely that a strong intrinsic signature can be obtained and used to measure total bacterial biomass in accordance with embodiments of the present invention. In other embodiments, the peak excitation wavelength may be from about 220 nm to about 240 nm or from about 270 nm to about 290 nm. Excitation-emission matrices shown for various bacteria and archaea and mineral particulates (see FIGS. 1 and 2) clearly indicate that ultraviolet laser induced fluorescence (UV LIF) is a powerful tool for reagentless discrimination between these two classes of particles.

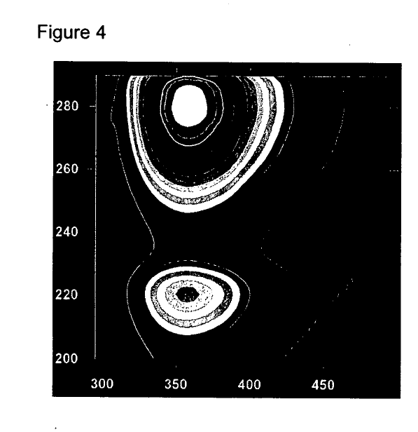

Tryptophan, Tyrosine and Phenylalanine are all intrinsically fluorescent. For tryptophan, the fluorescence emission reaches a maximum at 360 nm when the excitation is either ˜220 nm or ˜280 nm (see FIG. 4). For tyrosine, fluorescence peaks are observed at 300 nm as well as ˜415 nm particularly when excitation in the ˜230 nm or ˜275 nm wavelengths is provided (see FIG. 1).

The quantum yield of a fluorophore is the probability that a fluorescent photon produced per photons absorbed. The maximum extinction coefficient (epsilon max) indicates the capacity of the fluorophore to absorb light. Therefore, the “brightness” of a fluorophore is the product of the max extinction coefficient and the quantum yield. Molecule to molecule, tryptophan is nearly 6 times brighter than tryrosine and 140 times as bright as phenylalanine. Additionally, the extinction coefficient of tryptophan at 229 nm is even higher (13,000) than the published figure at 275 nm, giving it a brightness of 2600 if 229 nm excitation light is utilized.

TABLE 1

| | Trp | Tyr | Phe |
|---|---|---|---|
| λ_{emission max} (nm) | 348 | 303 | 282 |
| φ_{f} | 0.20 | 0.14 | 0.04 |
| τ_{f} (ns) | 2.6 | 3.6 | 6.4 |
| λ_{absorption max} (nm) | 280 | 274 | 257 |
| εₘₐₓ | 5600 | 1400 | 200 |
| εₘₐₓ · φ_{f} | 1120 | 196 | 8 |
| (brightness) | | | |

In one embodiment, if the number of events in a period of time exceeds a predetermined threshold, samples are collected by using the electrostatic sorting method discussed above or by using other methods such as a gated fluidics system. In embodiments wherein the sample is water that is presumably fairly pure, a rapid, dense sorting requirement may not be required. In other embodiments, (e.g., blood, plasma, etc.) rapid, dense sorting may be performed. As the microbial contaminants are collected in a container, they are concentrated prior to analysis. Once a sufficient concentration is reached, then other analytical methods can be used to identify the contaminant(s) present. These methods may include methods known in the art; for example, reverse-transcriptase polymerase chain reaction (RT-PCR), MALDI mass spectroscopy, traditional culturing methods, etc. One particular method, as described by U.S. Pub. No. 2005/0250141, herein incorporated by reference as though fully set forth in its entirely, uses a quantum-dot based lateral flow assay to identify microbial contaminants in typically less than five minutes using a simple test strip. Further, the inventive adaptive analytical method or immunoassay method as described herein may be used in accordance with embodiments of the present invention.

In one embodiment, if a predetermined threshold of microorganisms is exceeded, the cytometer deflects the microbial contaminant into a container, such as a test tube, a well or a cuvet. Various thresholds are applicable to various environments and purposes and thus the predetermined threshold will depend on the particular environment or purpose for which the system is used. By way of example, a 100 microoranisms/ml of non coliform bacteria may be a threshold for drinking water; and a threshold of 1 cfu of coliform bacteria may be a threshold for drinking water. The microorganism may then be iteratively incubated with spectrally multiplexed probes and analyzed by the cytometer to determine the phylogenetic lineage of the organism.

Methods of identifying contamination may require (sometimes significant) operator intervention. One way to alleviate this is to employ an in-place multiplexed assay to identify the microbial contaminant(s) using the flow cytometer itself in conjunction with a quantum dot immunoassay or an adaptive analysis method. Cadmium selenide quantum dots can all be excited by the same wavelength used to produce the intrinsic fluorescence of the microbial contaminants. Other chromophores and fluorophores do not share this property and most need separate light sources to excite each particular dye used in a multiplexed system. Specifically, the CdSe quantum dots all absorb ˜280 nm light and can be chosen (by their size) to fluoresce in spectrally narrow regions (e.g., about 30 nm) from 400-800 nm, which are far away from the intrinsic fluorescence emission of tryptophan in bacteria (˜320 nm). Additionally, quantum beads that use precise mixtures of CdSe of several colors to form spectral barcode labels may be used in embodiments of the present invention to run thousands of multiplexed tests in place with little or no operator intervention.

A number of probes (e.g., antibodies, aptamers, etc.) that bind specifically to particular strains of bacteria may be used for the immunoassay. A particular color of quantum dots or particular spectral bar code on a quantum bead may be bound to each type of antibody. These conjugates all fluoresce brightly but are all smaller than a wavelength of light so they do not scatter light as they pass through the cytometer. Therefore when they do not bind to their intended target, scattering does not occur. The unbound putative microbial concentrate fluoresces primarily from the tryptophan residues in the UV (˜320 nm) and scatters light. Therefore, when they do not bind to any labeled antibody they scatter light but do not strongly fluoresce in the visible region. Only when a quantum dot or a quantum bead tagged probe binds to the microbial contaminant do scatter and strongly visible fluorescence signatures simultaneously occur. When this occurs, the fluorescence signals and/or signatures may be used to determine the type of microorganism. As the detection occurs, the different fluorescence signals and/or signatures may be used to sort the microorganism and deflect different microorganisms into different containers, as described herein.

Another embodiment of the present invention provides for phylogenetically classifying a microorganism, such as a bacterium, via an adaptive tree-search algorithm using probes that detect ribosomal RNA. By determining lineage of an organism fewer probes are necessary to classify the vast number of possible microbial contaminants that could be present in a sample. In instances of monitoring biological threats, once lineage is determined, appropriate countermeasures for both engineered and known threats can be taken.

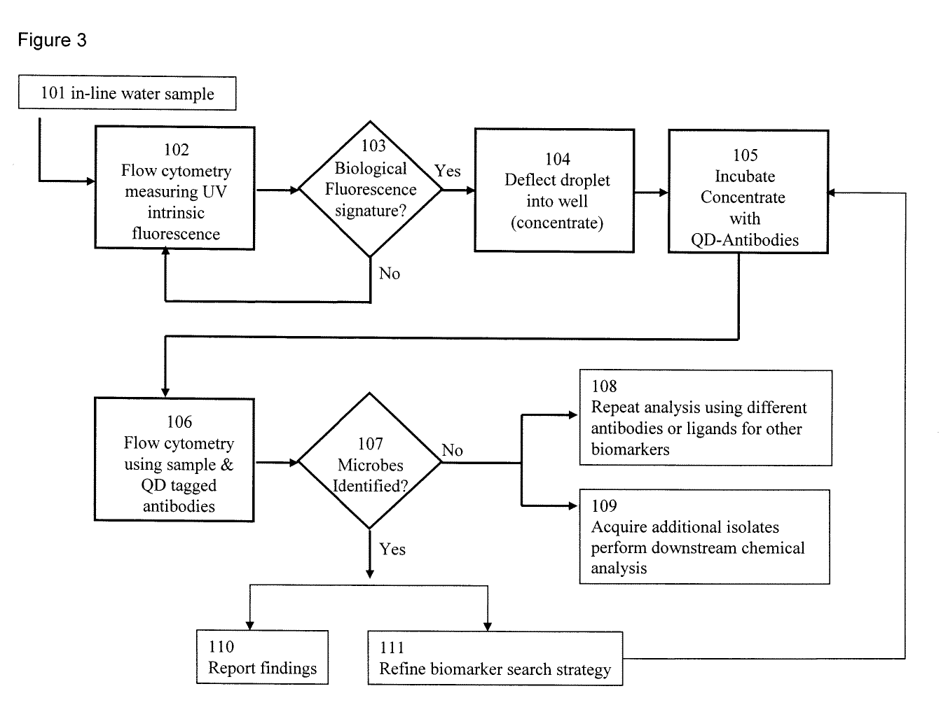

In one embodiment, as depicted in FIG. 3, a flow cytometer (or other fluidics apparatus or system) is used to determine events/second of simultaneous scattering and tryptophan intrinsic fluorescence. This is used as an automatic reagentless continuous indicator of biomass and identification of the biomass. In-line water sample 101 is placed through the flow cytometer to measure the UV intrinsic fluorescence 102, if a biological fluorescence signature is detected or if biomass events/sec exceeds a predetermined threshold 103, the particles are collected and concentrated for analysis by deflecting the water droplet containing the biomass into a well 104. After a sufficient concentration of the putative contaminant is obtained, the sample is then incubated with quantum dot-conjugated probes (e.g., antibodies, aptamers, PNAs, DNAs, RNAs, peptides) 105 and after binding can occur, it is placed through the flow cytometer again to identify the microorganism 106. This time, events with scatter and visible fluorescence are analyzed (for the spectral encoded ID of the microbe(s)). If the microorganism is not identified, additional concentrate can be obtained and tested by other instruments (e.g., RT-PCR, etc) 109 or additional quantum dot (QD) assays can be performed 108. If the microorganism is identified to a particular level (e.g., division, phylum, class, order, family, genus, species, subspecies), then the findings may be reported 110. Appropriate remediation can be performed from the findings. Otherwise, the biomarker search strategy is refined such that additional water droplets containing the microorganism are incubated with a set of probes that were synthesized in response to the identification of the microorganism at a particular level 111.

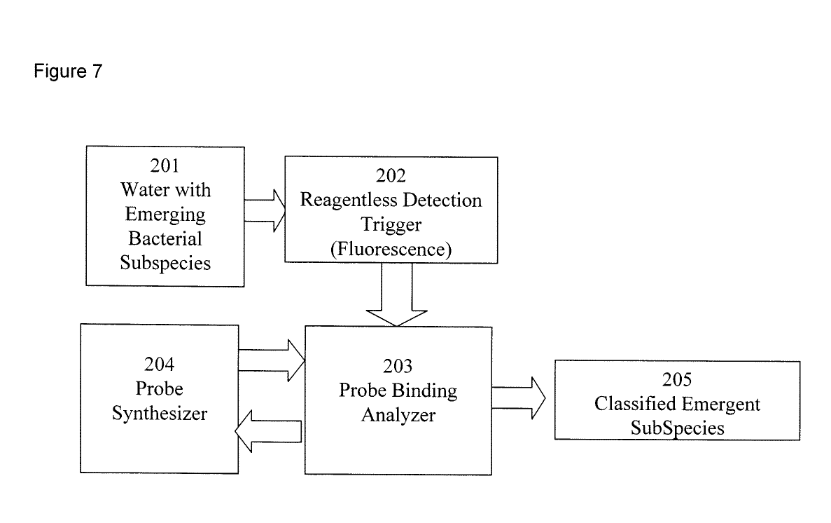

In another embodiment, as depicted in FIG. 7, water with emerging bacterial subspecies 201 is placed through a reagentless detection 202 to detect fluorescence of the bacterial subspecies. Once bacteria are detected the sample is placed in a probe binding analyzer 203 and the probe synthesizer 204 synthesizes probes based on information gained from the probe binding analyzer. After one or more iterations, the emergent subspecies of the bacteria is classified 205.

In one embodiment, the system is used for bacterial water monitoring. The system may provide a means for detecting and identifying known and emerging threats from biological agents for application in environmental monitoring systems as well as other monitoring systems. These threats can be man-made, from naturally occurring mutations, or induced from the environment (e.g., microgravity or radiation). The system may be used to determine the phylogeny of a bacterial strain (i.e., its evolutionary classification determined by its similarity to other species measured on a molecular level). It is known that bacterial 16S ribosomal RNA has sequences that have been conserved by the organism at different points in the evolutionary development of the organism. These sequences are routinely used to classify bacteria phylogenetically (division, phylum, class, order, family, genus, species, subspecies) using a large compiled 16S mRNA database. This system may be used to perform a step-wise tree-search analysis of the 16S ribosomal RNA genome using a flow cytometer (for analysis) and a probe synthesizer for on the fly probe fabrication. Because 500-60,000 ribosomes are present in bacteria, real-time cytometric molecular analysis can be performed without the need for PCR. By automatically determining the evolutionary relationship of an unknown bacterial strain with other known subspecies, one can (1) recognize that the bacteria is newly emergent or of a known type; and (2) develop better strategies to develop effective countermeasures against the organisms detected.

A continuous water monitoring system for bacterial contamination may use a two-tiered approach. A front end of this system may use UV light to reagentlessly detect microbial contamination in water. This front end system is used to trigger the phylogenetic classification when a given threshold of microbial contamination is present. A laser-induced fluorescence of intrinsic fluorophores in bacteria is used to continuously estimate the concentration of suspended biomass in the water supply being monitored. The system may alert users of the presence of the microbial load above established baseline thresholds without the use of reagents. If the threshold is exceeded, then an adaptive architecture may be employed for classification and/or identification of the contaminants.

If the inventive system measures a given counts/second biomass that exceeds a user-established threshold in biological particulates/second, the instrument may use electrostatic deflection to direct and concentrate the bacteria into a vessel for analysis using probes.

A flow cytometer may be used to detect and/or identify the bacteria; for example a 3 laser, 11 color, high-speed sorting flow cytometer (e.g., MoFlo™ available from Dako A/S; Denmark). The collection and excitation optics of the instrument may be designed to work across the UV and visible spectral regimes. In one embodiment, excitation wavelengths of about 229 nm and about 275 nm are used to induce fluorescence in bacteria interrogated by the instrument. Intrinsic fluorescence signatures, primarily due to tryptophan fluorescence at about 340 nm, along with laser induced forward and side scattering signatures is used for analyzing statistics regarding the presence of counting bacteria versus non-biological particulates. In other embodiments, other excitation wavelengths are used to induce fluorescence of the analyte. The excitation wavelength depends on the analyte that is being monitored or analyzed. One skilled in the art will be able to determine the appropriate excitation wavelength.

The inventive system may employ a synthesizer to produce multiplexed probes (e.g., PNA, DNA, RNA, aptamer, peptide, antibody probes) that are iteratively driven by an analyzer to identify all classes of biological threats (e.g., archaea, bacteria, viruses, prions, protein toxins) through a search algorithm examining the binding of various probes with the microbial contaminant or contaminants. The analyzer allows one to use information learned in one iteration to direct synthesis or selection of the appropriate probes for the next iteration. Phylogenetic classification of bacterial contaminants is just one example of the system's use. Ribosomal RNA phylogenetic analysis is one method that can be used to classify the type of bioagent(s) present in a water supply thereby enabling development of effective strategies to mitigate known and emerging threats.

In a particular embodiment, the system may use multiplexed peptide nucleic acid (PNA) probes to classify the bacteria by identification of a complementary set of 16S ribosomal ribonucleic nucleic acid (16S rRNA) sequences. PNA probes are more stable and bind their RNA targets better than their DNA analogs and like other oligonucleotides may be readily synthesized. In alternative embodiments, DNA, RNA, antibody, peptide or aptamer probes may be used. Typically, tens of thousand of ribosomes in bacteria contain 16S rRNA templates that provide intrinsic amplification of the hybridization signals, obviating the need for PCR.

16S rRNA sequences can be used to reveal not only the bacteria's species, but their lineage as well. For example, one 16S rRNA sequence is shared by every known bacterial strain on Earth. See e.g., Barrow et al, Cowan and Steel's Manual for the Identification of Medical Bacteria 3rd ed., Cambridge University Press (2001); Garrity et al., Bergey's Manual of Systematic Bacteriology Volume 1: The Archaea and the Deeply Branching and Phototrohic Bacteria, 2^{nd} ed. Springer (2001); Garrity et al., Bergey's Manual of Systematic Bacteriology Volume 2, Williams & Wilkins (1986), herein incorporated by reference as though fully set forth. Other sequences are unique for a bacteria's phylum, class, genus, species, subspecies, etc.

PNA probes are available or may be synthesized for sequences unique to a given position in the phylogenic tree of bacteria. See e.g., U.S. Patent Application. Publication 2001/0010910, U.S. Pat. No. 6,664,045, and U.S. Pat. No. 6,656,687, herein incorporated by reference as though full set forth. For example, microbes may be distinguished from the division of bacteria from archaea using a single PNA probe unique to the 16S rRNA sequences known to be present in all bacteria. Once the probe is tagged with a fluorophore and incubated with the sample, it penetrates the cells and hybridizes with the bacterial rRNA. The sample is then run through the cytometer again. If the PNA tag is bound within the microbe, the particle will both fluoresce (from the tag) and scatter light. If probe binding does not occur, the bacterial particles will scatter light, but not fluoresce (from the tag).

In subsequent iterations, hybridizations of other PNA probes complementary to rRNA sequences successively deeper in the phylogenetic tree are automatically examined. Fluorescently multiplexed PNA probes are used to determine template hybridization in parallel. The particular set of PNA sequences chosen for the next iteration will depend on the probes that hybridize in the previous iteration. An internationally complied phylogenic bacterial rRNA database may be used to guide the synthesis of each complementary PNA probe. Cole, J R et al. The Ribosomal Database Project (RDP-II): sequences and tools for high-throughput rRNA analysis. Nucleic Acids Res 2005 Jan. 1; 33(Database Issue):D294-D296. For example, after a particular phylum is identified, the system synthesizes probes that are complementary to specific classes within the phylum to identify the class to which the microorganism belongs. Once a class is identified, the system synthesizes probes that are complementary to specific orders within the class to identify the order to which the microorganism belongs. Once an order is identified, the system synthesizes probes complementary to specific families within the order to identify the family to which the microorganism belongs. Once a family is identified, the system synthesizes probes complementary to specific genera to identify the genus to which the microorganism belongs. Once the genus is identified, the system synthesizes probes complementary to specific species within the genus to identify the species to which the microorganism belongs. Once the species is identified, the system synthesizes probes that are complementary to specific subspecies within the species to identify the subspecies to which the microorganism belongs. This approach provides a means of classifying both known and unknown threats in terms of their evolutionary pathway(s), providing respondents with critical information as to the best countermeasures to employ to mitigate both natural and engineered biological threats.

The system may also be adapted for use as a field deployable device. Small Metal-Vapor Lasers and LEDs are available at 224 nm and 280 nm that correspond to the absorption bands of tryptophan. These sources used in conjunction with a suitable flow-through architecture are practical for field use.

### Adaptive Determination of Life Forms

The inventive system may also be used to determine and identify life forms on other terrestrial bodies (e.g., other planets and moons). The system may be adapted for use with robotic systems such as Mars exploration rovers and the like. The invention is not limited for use on any particular planet.

### Adaptive Determination of Cellular Chorography

In another embodiment, the system and method may be used to follow complex cellular chorography with a liquid based assay using a flow cytometer to examine binding with multiplexed probes in a mixture. The flow cytometer can therefore be viewed as a liquid biochip that analyzes probe binding within their cellular targets, the results of which are used to direct synthesis of new probes in a stepwise fashion to follow complex cellular processes automatically. Because both the assay and the synthesizer are programmable, multiple complex cellular processes can be followed in parallel.

Sometimes proteins encoded by the mRNA attach themselves to the DNA template and promote or suppress the expression of other genes. Other times, the proteins produced are enzymes that control chemical reactions that in turn cause other cascades of events controlling a cell's behavior or development. When such expression leads to a disease, scientists may design drugs (e.g., proteins or organic chemicals) to bind to specific mRNA templates or proteins to block a step in a complex pathway. Therefore, the inventive system and method have application to drug discovery, cancer research, proteomics, etc.

### Adaptive Production of Drugs Including Aptamers

In one embodiment of the present invention, the system is used to produce aptamers that are tailored to specific pathogens (e.g., cancer cells via their surface markers) corresponding to the changes and mutation of the pathogen in the body.

At birth there are a large number of antibodies circulating at low levels. Once an antibody recognizes and attaches to a pathogen, a reaction occurs whereby many other clones of that particular antibody as well as memory cells are made. The memory cells become dormant factories of the given antibody and upon a second exposure, are activated (e.g., chicken pox). There are mutations that occur in the memory cells however, which cause the population of antibodies made during a second exposure to be more diverse than the original antibody that recognized and attached to the pathogen during the first exposure. These other variants of a particular antibody circulate and it is more likely that one of these variants has a higher affinity and/or specificity than the original antibody. This process continues and thus the body is always “tuning” or adapting its sensors (i.e., producing more specific antibodies or variant antibodies) to allow them to adapt to the latest strain of the pathogen. Since aptamers can be synthetic antibodies made from nucleic acids or peptides, the present inventive system may be used to adapt as well, and make the appropriate aptamers for the situation. For example, an aptamer is made to recognize, attach and/or treat to a particular type of cancer. The present inventive system may be used to monitor the cancer cell surface markers and make additional aptamers which are tailored to the cancer cell surface markers as they change and mutate while growing in the body.

In another embodiment, more specific aptamers may be made by the adaptive process of the present invention. By way of example, a cell surface marker may be the target (i.e., the analyte) for which an aptamer is made. An initial set of fluorophore conjugated aptamers may be made to bind the cell surface marker. Different fluorophores may be conjugated to different aptamers. These aptamers are incubated with the cell surface marker and then placed through the system where the binding information is gained. Of these aptamers some may bind and some may not bind. Information regarding the binding is used to synthesize additional aptamers that may have different and/or more specific binding to the cell surface marker. For example, the aptamers that bind to the cell surface maker may comprise similar sequences and thus the computer uses this information to synthesize aptamers comprising the similar sequences but with additional sequences that differ from the initial or previous set of aptamers. The process may be repeated until one or more desirable aptamers are synthesized.

Embodiments of the present invention are not limited to aptamers as DNA, RNA, peptides, proteins, chemicals, chemical compounds, etc., may be used in place of the aptamers. Embodiments of the present invention are also not limited to cell surface markers, as other targets may be the analyte for which a therapeutic compound is produced by the inventive method and system.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Alfipia, Burkolderia, and Rhodopseudomonas are bacterial strains that were cultured from soil obtained from the dry valley of the Atacama Desert in Chile. Halobacteria salinarum, Deinococcus radiodurans, and Psychrobacter cryopegella have adapted to conditions of high salinity, dryness & radiation, and cold temperatures, respectively. Magnetosomes (intracellular magnetite crystals) in AMB-1 cause them to be ferromagnetic. Synechocystzs sp. are photosynthetic oxygen producing cyanobacteria and in the absence of light, can survive by utilizing energy and carbon from an appropriate source (e.g. glucose). All of these microorganisms have a dominant tryptophan component in their EEM (EEM's of Synechocystis sp. and Halobacteria also show the presence of tyrosine). See FIG. 1.

While metal ion impurities can change the appearance of EEM signatures, none of these signatures, except nominally pyroxene and magnetite (both minimally fluorescent, different peak position), are similar to the signature of tryptophan. See FIG. 2. Therefore, from EEM data of all extremophiles tested to date, tryptophan is a compelling choice as the universal intrinsically fluorescent biomarker.

TABLE 2

| | Peak Excitation | Peak Emission |
|---|---|---|
| Compound | Wavelength (nm) | Wavelength (nm) |
| Tryptophan | 220, 280 | 357 |
| NADH | 360 | 450 |
| F420 | 420 | 470 |
| Chlorophyll-a | Many | 685 |
| Favins | 460 | 540 |
| PAHs | 300 | 450 |

### Example 2

Reagentless detection. A large frame argon laser and a frequency doubled argon laser are tuned to 275 nm and 229 nm, respectively. These wavelengths are used to interrogate the hydrodynamically focused fluid stream containing the sample. Forward and side scatter data from a Krypton laser (407 nm) and intrinsic fluorescence from the bacteria (340 nm) in the stream is collected by photomultipliers and used to provide a reagentless detection of single bacteria as they flow by. Photomultipliers may also be used to measure fluorescence in the 330 nm regime using separate spots on the sample flow stream where 229 nm and 275 nm light are applied using a frequency doubled and large frame UV argon laser respectively. If a predetermined threshold is exceeded, then the bacteria are collected from the stream by electrostatically deflecting them into a test tube. Alternatively, the bacteria/second detection rate is used to determine if a predetermined threshold (e.g., a threat threshold) has been reached (e.g., a sudden increase over background).

Phylogenetic classification. Four colors of quantum dots that fluoresce at 565 nm, 605 nm, 655 nm, and 705 nm are conjugated to four different antibodies that are selected to bind to different epitopes of four different strains of bacteria. The 275 nm excitation beam will be used to excite all four quantum dot conjugates after being incubated with test samples.

Alternatively, previously synthesized PNA probes at the top of the bacterial phylogenic tree that have been conjugated to CdSe quantum dots are incubated with the bacteria collected. Upon incubation the PNA probes enter the cells and bind to any complementary RNA templates in the thousands of ribosomes in the bacteria in the sample. An argon laser operating at 352 nm is used to excite the family of quantum dots. Visible emission with concomitant 407 nm scatter indicates PNA binding. Additional bacteria are collected and new PNA probe conjugates are added on the branch of the phylogenetic tree determined in the previous round. These classification steps continue until the bacteria in the sample have been phylogenetically classified to the desired level; for example, the subspecies level.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for monitoring an analyte in a sample, comprising:
providing the sample; and
detecting a presence of the analyte in the sample,
wherein detecting the presence of the analyte comprises:
placing the sample through a fluidics apparatus,
using a laser to excite an intrinsic fluorophore in the analyte,
detecting a fluorescence in the sample, and
comparing the detected fluorescence to a database of information,
including information on the fluorescence of the intrinsic fluorophore in the analyte,
wherein if the detected fluorescence corresponds to the fluorescence of the intrinsic fluorophore, the analyte is determined to be present in the sample.

2. The method of claim 1, wherein the fluidics apparatus is a flow cytometer.

3. The method of claim 1, wherein the laser operates at a wavelength of from about 220 nm to about 240 nm and/or from about 270 nm to about 290 nm, and detecting the fluorescence comprises detecting a fluorescence signal of from about 320 nm to about 370 nm.

4. The method of claim 1, further comprising collecting the analyte by electrostatically deflecting a portion of the sample containing the analyte into a container.

5. The method of claim 1, further comprising analyzing the analyte.

6. The method of claim 5, wherein analyzing the analyte comprises using a fluorophore-conjugated immunoassay method or a fluorophore-based adaptive analysis method.

7. The method of claim 6, wherein the fluorophore is a quantum dot or a quantum bead.

8. The method of claim 6, wherein the immunoassay method comprises:
using a fluorophore-conjugated probe capable of binding to a particular analyte; and
determining the binding of the fluorophore-conjugated probe to the analyte,
wherein the binding of the fluorophore-conjugated probe to the analyte indicates the presence of the particular analyte.

9. The method of claim 8, wherein determining the binding of the fluorophore-conjugated probe comprises:
using a laser at a wavelength capable of exciting the fluorophore; and
detecting the emission of the fluorophore and a scattering of light by the analyte,
wherein the detection of both the emission of the flurorphore and the scattering of the light indicate the binding of the probe to the analyte.

10. The method of claim 8, wherein the probe is an antibody or an aptamer.

11. The method of claim 6, wherein the fluorophore-based adaptive analysis method comprises:
providing an initial fluorophore conjugated probe set;
adding the probe set to the sample;
determining the binding of the probe to the analyte to produce binding results;
comparing the binding results to a database of information regarding the analyte;
generating a new fluorophore conjugated probe set based on the comparison; and
repeating the process with the new fluorophore conjugated probe set until the desired analysis of the analyte is performed.

12. The method of claim 11, wherein the probe is selected from the group consisting of deoxyribonucleic acid (DNA), ribonucleic acid (RNA), peptide nucleic acid (PNA), aptamer, peptide, antibody and combinations thereof.

13. The method of claim 11, wherein determining the binding of the probe to the analyte comprises:
using a laser at a wavelength capable of exciting the fluorophore; and
detecting the emission of the fluorophore and a scattering of light by the analyte,
wherein the detection of both the emission of the fluorophore and the scattering of the light indicate the binding of the probe to the analyte.

14. The method of claim 1, wherein the analyte is a microorganism or a cell.

15. The method of claim 1, wherein the sample is water or a physiological fluid.

16. A method for adaptive analysis of an analyte in a sample, comprising:
providing an initial fluorophore conjugated probe set;
adding the probe set to the sample;
determining the binding of the probe to the analyte to produce binding results;
comparing the binding results to a database of information regarding the analyte;
generating a new fluorophore conjugated probe set based on the comparison; and
repeating the process with the new fluorophore conjugated probe set until the desired monitoring or analysis of the analyte is performed.

17. The method of claim 16, wherein the probe is selected from the group consisting of deoxyribonucleic acid (DNA), ribonucleic acid (RNA), peptide nucleic acid (PNA), aptamer, peptide, antibody and combinations thereof.

18. The method of claim 16, wherein the probe is a peptide nucleic acid (PNA).

19. The method of claim 16, wherein the probe is an aptamer.

20. The method of claim 16, wherein the fluorophore is a quantum dot or a quantum bead.

21. The method of claim 16, wherein determining the binding of the probe to the analyte comprises:
using a laser at a wavelength capable of exciting the fluorophore; and
detecting the emission of the fluorophore and a scattering of the light by the analyte,
wherein the detection of both the emission of the fluorophore and the scattering of the light indicate the binding of the probe to the analyte.

22. The method of claim 16, wherein the analyte is a microorganism or a cell.

23. The method of claim 16, wherein the sample is water or a physiological fluid.

24. A system for monitoring an analyte in a sample, comprising:
a fluidics apparatus adapted to create a fluid stream of the sample and/or to create a series of small drops of the sample;
a laser adapted to operate at a wavelength that is capable of inducing an intrinsic fluorescence of the analyte;
a counting component adapted to determine the concentration of the analyte;
an reporting component adapted to report the concentration of analyte that is above or below a predetermined concentration; and
a sorting component adapted to apply a charge to a portion of the sample containing the analyte and to deflect the charged portion of the sample containing the analyte into a container.

25. The system of claim 24, wherein the fluidics apparatus is a flow cytometer.

26. The system of claim 24, wherein the analyte is a microorganism or a cell.

27. An adaptive analysis system for analysis of an analyte in a sample, comprising:
a fluidics apparatus adapted to determine the binding of a fluorophore conjugated probe to the analyte;
a probe synthesizer to synthesize a new set of fluorophore conjugated probes; and
a computer adapted to analyze the binding of the probes, compare the binding of the probes to a database of information regarding the analyte, and provide information to the probe synthesizer regarding the type of probes to synthesize.

28. The system of claim 27, wherein the analyte is a microorganism or a cell.

29. The system of claim 27, wherein the probe is selected from the group consisting of deoxyribonucleic acid (DNA), ribonucleic acid (RNA), peptide nucleic acid (PNA), aptamer, peptide, antibody and combinations thereof.

30. The system of claim 27, wherein the fluorophore is a quantum dot or a quantum bead.

31. A method of phylogenetic classification of a microorganism in a sample, comprising:
providing an initial fluorophore conjugated probe set that is complementary to a 16S ribosomal ribonucleic acid (rRNA) sequence;
adding the probe set to the sample;
determining the binding of the probe to rRNA in the microorganism to generate binding results;
comparing the binding results to a database of information regarding microorganisms;
generating a new fluorophore conjugated probe set based on the comparison; and
repeating the process with the new fluorophore conjugated probe set until the desired level of phylogenetic classification of the microorganism is performed.

32. The method of claim 31, wherein the probe is a peptide nucleic acid (PNA).

33. The method of claim 31, wherein the fluorophore is a quantum dot or a quantum bead.

34. A method for adaptive production of a therapeutic compound, comprising:
providing an initial set of fluorophore conjugated therapeutic compounds capable of binding to an analyte for which the therapeutic compound is to be produced;
adding the set of fluorophore conjugated therapeutic compounds to a sample comprising the analyte;
determining the binding of the therapeutic compounds to the analyte to generate binding results;
comparing the binding results to determine a similarity between bound therapeutic compounds and/or comparing the binding results to a database of information regarding the analyte;
generating a new set of fluorophore conjugated therapeutic compounds based on the comparison; and
repeating the process with the new set of fluorophore conjugated therapeutic compounds until the desired therapeutic compound is synthesized.

35. The method of claim 34, wherein the therapeutic compound comprises deoxyribonucleic acid (DNA), ribonucleic acid (RNA), peptide nucleic acid (PNA), aptamer, peptide, antibody or combinations thereof.

36. The method of claim 34, wherein the fluorophore is a quantum dot or a quantum bead.
